(19) United States
(12) Patent Application Publication
(10) Pub. No.: US 2007/0225379 A1
(43) Pub. Date: Sep. 27, 2007

(54) TRANSDERMAL DELIVERY OF SYSTEMICALLY ACTIVE CENTRAL NERVOUS SYSTEM DRUGS

(76) Inventors: Dario Norberto R. Carrara, Oberwil (CH); Arnaud Grenier, Steinbrunn-Le-Haut (FR); Igno Alberti, Huningue (FR); Laetitia Henry, Saint Louis (FR); Celine Decaudin, Saint Louis (FR)

Correspondence Address: WINSTON & STRAWN LLP;PATENT DEPARTMENT, 1700 K STREET, N.W., WASHINGTON, DC, 20006, US

(21) Appl. No.: 11/755,923

(22) Filed: May 31, 2007

Related U.S. Application Data
(63) Continuation-in-part of application No. 11/371,042, filed on Mar. 7, 2006.
Application No. 11/371,042 is a continuation of application No. PCT/EP04/11175, filed on Oct. 6, 2004.
(63) Continuation-in-part of application No. 11/634,005, filed on Dec. 4, 2006.
Application No. 11/634,005 is a continuation of application No. 10/343,570, filed on May 19, 2003, now Pat. No. 7,214,381.
(60) Provisional application No. 60/510,613, filed on Oct. 10, 2003.

(51) Int. Cl. A61K 47/00; A61K 31/025

(52) U.S. Cl. 514/756; 514/772

(57) ABSTRACT

The invention relates to a transdermal or transmucosal non-occlusive, semi-solid pharmaceutical formulation that includes at least one systemically active agent that acts on the Central Nervous System (CNS) of a mammal; and a permeation enhancing solvent system present in an amount sufficient to solubilize the at least one active ingredient. The permeation enhancing solvent system includes a pharmaceutically acceptable monoalkyl ether of diethylene glycol; a pharmaceutically acceptable glycol; preferably also a fatty alcohol and or a fatty acid; and a mixture of a C₂ to C₄ alcohol and water so that the permeation enhancing solvent system (a) inhibits crystallization of the at least one active ingredient on a skin or mucosal surface of a mammal, (b) reduces or prevents transfer of the formulation to clothing or to another being, (c) modulates biodistribution of the at least one active agent within different layers of skin, (d) facilitates absorption of the at least one active agent by a skin or a mucosal surface of a mammal, or (e) provides a combination of one or more of (a) through (d).

### CROSS REFERENCE TO RELATED APPLICATIONS

This application is a continuation-in-part of U.S. patent application Ser. No. 11/371,042 filed Mar. 7, 2006, which is a continuation of International application no. PCT/EP2004/011175 filed Oct. 6, 2004, which claims the benefit of U.S. provisional patent application No. 60/510,613, filed Oct. 10, 2003. This application is also a continuation-in-part of U.S. patent application Ser. No. 11/634,005 filed Dec. 4, 2006, which is a continuation of application Ser. No. 10/343,570 filed May 19, 2003, now U.S. Pat. No. 7,214,381, which is the U.S. national stage of International application no. PCT/EP01/09007 filed Aug. 3, 2001.

### FIELD OF THE INVENTION

The present invention relates to a novel transdermal or transmucosal pharmaceutical formulation comprising at least one active ingredient and a solvent system. The invention reveals a pharmaceutical formulation that administers the active drug(s) at a permeation rate that would ensure therapeutically effective systemic concentration. The formulations of the present invention contain defined amounts of chemicals that minimize the barrier characteristics of the most uppermost layer of the epidermis and provide sustained permeation rate. Said chemicals are fatty alcohols such as n-decanol, lauryl alcohol, myristyl alcohol, oleyl alcohol, etc., diethylene glycol monoethyl ether and propylene glycol solubilized in a hydro-alcoholic binary vehicle composite. The invention also relates to a method of delaying or inhibiting crystallization of an active agent in a transdermal or transmucosal pharmaceutical formulation.

### BACKGROUND OF THE INVENTION

It is well known that many drugs taken orally are destroyed on the first pass through the liver. It is also well known that when many drugs are taken orally, their rate of absorption into the body is not constant. In view of such difficulties, a number of different drug delivery systems have been developed.

The transdermal or transmucosal route for delivery of drugs provides many advantages, and transdermal or transmucosal systems for delivering a wide variety of drugs are described in U.S. Pat. Nos. 5,785,991; 4,764,381; 4,956,171; 4,863,970; 5,453,279; 4,883,660; 5,719,197 or EP patent application number 0 271 983; 0 267 617; 0 261 429; 0 526 561; as an example, some of which are mentioned hereinafter.

A major drawback of this therapy however is the limitation of the amount of drug that can be transported across the skin. In many cases, drugs which would appear to be ideal candidates for transdermal delivery are found to have such low permeability through intact skin that they cannot be delivered in therapeutically effective amounts from transdermal devices. This limitation is due to several factors. Since the skin is a protective barrier by nature, the rates of transport of most compounds through the skin are quite slow. It is generally accepted that a surface of patch beyond 50 square centimeters would result in difficulty of application. Therefore the application of a transdermal semisolid dosage form such as a gel, cream, ointment, liquid, etc., augments the patient's compliance and the surface of application can be extended.

In order to increase skin permeability so that drugs can be delivered in therapeutically effective amounts at therapeutically effective rates, it has been proposed different systems or devices or mechanisms one of which is deliver the drug(s) in presence of permeation enhancers. Usually, using penetration enhancing compounds, processes or devices to increase drug penetration solve this problem.

Various systems were suggested for this purpose, as described in different patents such as U.S. Pat. Nos. 5,785,991; 4,764,381; 4,956,171; 4,863,970; 5,453,279; 4,883,660; 5,719,197 or W.O. patents No. 97/29735; 98/17316 or in the literature “Pharmaceutical Skin Penetration Enhancement”, J. Hadgraft, Marcel Dekker, Inc. 1993; “Percutaneous Absorption”, R. Bronaugh, H. Maibach, Marcel Dekker, Inc. 1989, etc.

To be accepted, a permeation enhancer or a combination thereof should have the ability to enhance the permeability of the skin for the drug, should be non-toxic, non-irritant and non-sensitizing on repeated exposure.

It is often difficult to predict which compounds will work as permeation enhancers and which permeation enhancers will work for particular drugs. In transdermal drug delivery applications, a compound that enhances the permeability of one drug or a family of drugs may not necessarily enhance the permeability of another drug or family of drugs. That is also concluded after careful analysis of the scientific literature relating to this specific topics, such as “Transdermal Therapeutic Systemic Medications, Marcel Dekker Inc., New York, 1989” (see table on page 3).

Therefore, the usefulness of a particular compound(s) or mixture thereof as a permeation enhancer must be carefully analyzed and demonstrated by empirical work.

EP 0 279 977 describes a transdermal device for administering progesterone and an estradiol ester alone or in combination, utilizing a polymer matrix which has the drug(s) with a penetration enhancer such as sucrose monococoate, glycerol monooleate, sucrose monolaurate, glycerol monolaureate, etc.

EP 0 367 431 discloses that aliphatic alcohols such as isopropyl alcohol and isobutyl alcohol that are commonly used in topical transdermal formulation, thus, enhance the rate of transdermal delivery of steroid drugs.

EP 0 526 561 relates to the use of chemical penetration enhancers to enhance the transdermal delivery of medicaments through the skin, said chemical enhancers are alcohols.

WO 90/11 064 discloses a skin penetration enhancer composition for transdermally administered pharmacologically active agents. The composition contains diethylene glycol monoethyl or monomethyl ether in addition to an ester component such as propylene glycol monolaurate, methyl laurate or the like.

U.S. Pat. No. 4,764,381 describes pharmaceutical preparations comprised of a pharmaceutically active ingredient and a carrier which comprises a percutaneous penetration enhancer comprised of 2-ethyl-1,3 hexanediol alone or in combination with oleic acid.

U.S. Pat. No. 4,863,970 discloses penetration-enhancing pharmaceutical compositions for topical transepidermal and percutaneous application which are non-irritating to the skin and describes a binary system of oleic acid or alcohol and a lower alcohol.

U.S. Pat. No. 4,973,468 describes to skin permeation enhancer compositions which increase the permeability of skin to transdermally administered pharmacologically active agents. The composition contains diethylene glycol monoethyl or monomethyl ether in addition to an ester component such as propylene glycol monolaurate, methyl laurate or the like.

U.S. Pat. No. 5,325,457 discloses a transdermal occlusive patch composition comprising a balance between volatile and non-volatile solvents to create systems ensuring supersaturation of drugs.

U.S. Pat. No. 5,453,279 describes an enhancing transdermal absorption composition useful in transdermal absorption of progestins including progesterone and optionally an estrogen for contraceptive or HRT. The enhancing composition comprises a combination of a lower alkyl ester of a polycarboxylic acid, an aliphatic monohydroxy alcohol and an aliphatic diol.

U.S. Pat. No. 5,580,574 discloses a transdermal occlusive patch composition consisting essentially of a benzodiazepine or a benzodiazepine antagonist, isopropanol, propylene glycol, oleic acid and water as the essential permeation enhancers. Propylene glycol is recited in amounts ranging from 30 to 50% wt amounts. There is no disclosure that diethylene glycol monoethyl ether or any other diethylene glycol monoalkyl ether as an essential ingredient in order to obtain enhanced performance such as the avoidance of drug crystallization.

U.S. Pat. No. 5,658,587 describes transdermal therapeutic systems for the delivery of alpha adrenoceptor blocking agents using a solvent enhancer system comprising diethylene glycol monoethyl ether and propylene glycol.

U.S. Pat. No. 5,662,890 discloses an alcohol-free cosmetic composition for artificially tanning the skin containing a combination of diethylene glycol monoethyl ether and dimethyl isosorbide as permeation enhancer.

U.S. Pat. No. 5,785,991 describes a composition, device and method for transdermal administration of an active agent using a novel dual permeation enhancer mixture comprising lauryl acetate and a monoglyceride, glycerol monolaurate.

U.S. Pat. No. 5,891,462 discloses a pharmaceutical formulation in the form of a gel suitable for the transdermal administration of an active agent of the class of estrogens or of progestin class or of a mixture thereof, comprising lauryl alcohol, diethylene glycol monoethyl ether and propylene glycol as permeation enhancers.

U.S. Pat. No. 5,932,243 describes a pharmaceutical emulsion or microemulsion preconcentrate for oral administration of macrolide containing a hydrophilic carrier medium consisting of diethylene glycol monoethyl ether, glycofurol, 1,2-propylene glycol, or mixtures thereof.

U.S. Pat. Nos. 6,267,985 and 6,383,471 disclose pharmaceutical compositions and methods for improved solubilization of triglycerides and improved delivery of therapeutic agents containing diethylene glycol monoethyl ether and propylene glycol as solubilizers of ionizable hydrophobic therapeutic agents.

U.S. Pat. No. 6,929,801 describes a transdermal drug delivery system comprising a therapeutically effective amount of an anti-Parkinson agent such as pramipexole, at least one dermal penetration enhancer which is a skin-tolerant ester sunscreen, and at least one volatile liquid.

U.S. Pat. No. 6,426,078 discloses an oil-in water microemulsion containing diethylene glycol monoethyl ether or propylene glycol as co-emulsifier of lipophilic vitamins.

Many research experiments have been carried out on diethylene glycol monoethyl ether (marketed under the trademark TRANSCUTOL™ by Gattefosse) as an intracutaneous drug depot builder. For example, Ritschel, W. A., Panchagnula, R., Stemmer, K., Ashraf, M., “Development of an intracutaneous depot for drugs. Binding, drug accumulation and retention studies, and mechanism depot for drugs”, Skin Pharmacol, 1991; 4: 235-245; Panchagnula, R. and Ritschel, W. A., “Development and evaluation of an intracutaneous depot formulation of corticosteroids using TRANSCUTOL® as a cosolvent, in vitro, ex vivo and in-vivo rat studies”, J. Pharm. Pharmacology. 1991; 43: 609-614; Yazdanian, M. and Chen, E., “The effect of diethylene glycol monoethyl ether as a vehicle for topical delivery of ivermectin”, Veternary Research Com. 1995; 19: 309-319; Pavliv, L., Freebern, K., Wilke, T., Chiang, C- C., Shetty, B., Tyle, P., “Topical formulation development of a novel thymidylate synthase inhibitor for the treatment of psoriasis”, Int. J. Pharm., 1994; 105: 227-233; Ritschel, W. A., Hussain, A. S., “In vitro skin permeation of griseofulvin in rat and human skin from an ointment dosage form”, Arzneimeittelforsch/Drug Res. 1988; 38: 1630-1632; Touitou, E., Levi-Schaffer, F., Shaco-Ezra, N., Ben-Yossef, R. and Fabin, B., “Enhanced permeation of theophylline through the skin and its effect on fibroblast proliferation”, Int. J. Pharm., 1991; 70: 159-166; Watkinson, A. C., Hadgraft, J. and Bye, A., “Enhanced permeation of prostaglandin E₂ through human skin in vitro”, Int. j. Pharm., 1991; 74: 229-236; Rojas, J., Falson, F., Courraze, G., Francis, A., and Puisieux, F., “Optimization of binary and ternary solvent systems in the percutaneous absorption of morphine base”, STP Pharma Sciences, 1991; 1: 71-75; Ritschel, W. A., Barkhaus, J K., “Use of absorption promoters to increase systemic absorption of coumarin from transdermal drug delivery systems”, Arzneimeittelforsch/Drug Res. 1988; 38: 1774-1777.

None of the above mentioned inventions or publications report a study of a permeation enhancing system of propylene glycol together with diethylene glycol monoethyl ether and fatty alcohols or fatty acids in a binary hydro-alcoholic vehicle composite in a semi-solid dosage form, designed to administer transdermally or through the mucosal membrane the group of active agents mentioned in the present invention. None of the above mentioned inventions or publications describes an adequate transdermal or transmucosal formulation to deliver therapeutic plasma levels of different types of active compounds, as is now disclosed by the present invention.

### SUMMARY OF THE INVENTION

The composition of the present invention relates to a penetration enhancing system that can be utilized in many types of products for topical or transdermal application, that include, but are not limited to, solutions, creams, lotions, sprays, ointment, gels, aerosols and patch devices.

The transdermal or transmucosal pharmaceutical formulation of the present invention comprises at least one active agent; and a solvent system present in an amount sufficient to solubilize the at least one active ingredient and to inhibit crystallization of the at least one active ingredient on a skin or mucosal surface of a mammal. Other advantages of the transdermal or transmucosal pharmaceutical formulation of the invention include reducing or preventing the transfer of the formulation to clothing or another, minimizing contamination of clothing by the formulation, modulating biodistribution of the active agent(s) within the different layers of the skin, and facilitating absorption of the active agent(s) by the skin or mucosa surface to name a few.

The novel permeation enhancing system of the present invention includes a solvent system comprising a fatty alcohol, a fatty acid, or mixtures thereof, a diethylene glycol monoalkyl ether, present in an amount of between about 1% and 30% by weight of the solvent system, and a glycol, present in an amount of between about 1% and 30% by weight of the solvent system. The diethylene glycol monoalkyl ether and glycol are present in preferred weight ratios of 10:1 to 1:10. The solvent system further includes a mixture of an alcohol and water. This hydro-alcoholic mixture is present in an amount of between about 40% and 98% of the solvent system, wherein the alcohol is present in an amount of about 5% to 80% of the mixture, and the water is present in an amount of about 20% to 95% of the mixture.

Surprisingly, it has been discovered that the combinative use of a monoalkyl ether of diethylene glycol and a glycol at specified ratios, preferably in presence of a fatty alcohol and/or a fatty acid, and preferably also in hydro-alcoholic compositions, prevents or significantly reduces the transfer of active drug(s) from transdermal semi-solid formulations to clothing or other surfaces; significantly reduces the transfer to individuals; and also prevents or significantly reduces the loss of active drug(s)—and therefore the loss of therapeutic efficiency—consecutive to accidental removing due to daily activities such as washing, swimming or the like.

Other advantages of the present invention include the discovery that the association of a monoalkyl ether of diethylene glycol and a glycol at specified ratios exhibits a synergic effect and inhibits crystallization of the active ingredient(s) in transdermal semi-solid formulations. In addition, it has been discovered, against the background described above, a totally unexpected control of the active drug(s) distribution in the different layers of the skin is achieved when modifying the range of the monoalkyl ether:glycol ratio described in the present invention, simultaneously but independently from the crystallization inhibitor effect above mentioned.

Further, it has also been found that the glycol acts as a modulator of the capability of the monoalkyl ether of diethylene glycol to build a drug depot within the different layers of the skin. Also, the significant reduction of unabsorbed active drug(s) remaining at the application surface area results from the simultaneous although independent inhibition of crystallization and transdermal drug penetration, enhanced or not by additional permeation enhancer(s).

In preferred embodiments, the composition further comprises a gelling agent and a pH-adjusting agent whenever deemed necessary. In preferred embodiments, the gelling agent is a carbomer (polyacrylic acid) or a cellulose derivative, and the pH-adjusting agent is an organic amine like monoethanolamine, diethanolamine, triethanolamine, tromethamine, or diisopropylamine. Pharmaceutical compounding agents such as, but not limited to, preservatives, anti-oxidants, chelatants, flavors, fragrances, sweeteners, film forming agents, solubilizers, etc. . . . can be added as well. In preferred embodiments, the composition is non occlusive. The penetration enhancing system of the present invention can also be used for mucosal delivery through the buccal, sublingual, auricular, nasal, ophthalmic, rectal, or vaginal mucosa.

Hence, it has been surprisingly discovered that it is possible to achieve a therapeutically effective, sustained and controlled penetration rate of diverse active substances into the skin with the aid of the inventive means. It has also been discovered surprisingly that the compositions disclosed herein exert higher permeation rates when compared with compositions that do not contain the invention.

It has been surprisingly discovered also that by utilizing diethylene glycol monoethyl ether, propylene glycol, and also preferably decyl, lauryl or myristyl alcohol, as permeation enhancing system for the invention herein disclosed, an adequate penetration enhancement factor and a sustained flux of the active agent is attained, thereafter reflected in achieving therapeutic effective, controlled and sustained levels of the active drugs by only once-a-day application of the formulation.

Thus, the present invention relates to a method for administering topically or systemically different active substance(s).

### BRIEF DESCRIPTION OF THE FIGURES

The features of the invention will be further described in the following detailed description and accompanying drawings in which:

FIG. 1A graphically illustrate the effect of the present invention on the 24-hour cumulative drug permeation of anti-Parkinson drug pramipexole;

FIG. 1B graphically illustrate the effect of the permeation enhancing system of the present invention on the drug instant flux of anti-Parkinson drug pramipexole;

FIG. 2 graphically illustrate the effect of the permeation enhancing system of the present invention on the 24-hour cumulative drug permeation of anti-Parkinson drug pramipexole;

FIG. 3A graphically illustrate the effect of the permeation enhancing system of the present invention on the 24-hour cumulative drug permeation of anti-Alzheimer drug rivastigmine;

FIG. 3B graphically illustrate the effect of the permeation enhancing system of the present invention on the drug instant flux of anti-Alzheimer drug rivastigmine;

FIG. 4A graphically illustrate the effect of the permeation enhancing system of the present invention on the 24-hour cumulative drug permeation of analgesic drug fentanyl;

FIG. 4B graphically illustrate the effect of the permeation enhancing system of the present invention on the drug instant flux of analgesic drug fentanyl;

FIG. 5A graphically illustrate the effect of the permeation enhancing system of the present invention on the 24-hour cumulative drug permeation of analgesic drug fentanyl;

FIG. 5B graphically illustrate the effect of the permeation enhancing system of the present invention on the drug instant flux of analgesic drug fentanyl;

FIG. 6A graphically illustrate the effect of the permeation enhancing system of the present invention on the 24-hour cumulative drug permeation of anti-Parkinson drug selegiline;

FIG. 6B graphically illustrate the effect of the permeation enhancing system of the present invention on the drug instant flux of anti-Parkinson drug selegiline;

FIG. 7A graphically illustrate the effect of the permeation enhancing system of the present invention on the 24-hour cumulative drug permeation of anti-Parkinson drug pergolide;

FIG. 7B graphically illustrate the effect of the permeation enhancing system of the present invention on the drug instant flux of anti-Parkinson drug pergolide;

FIG. 8A graphically illustrate the effect of the permeation enhancing system of the present invention on the 24-hour cumulative drug permeation of anti-Parkinson drug ropinirole;

FIG. 8B graphically illustrate the effect of the permeation enhancing system of the present invention on the drug instant flux of anti-Parkinson drug ropinirole;

FIG. 9A graphically illustrate the effect of the permeation enhancing system of the present invention on the 24-hour cumulative drug permeation of anti-Parkinson drug ropinirole;

FIG. 9B graphically illustrate the effect of the permeation enhancing system of the present invention on the drug instant flux of anti-Parkinson drug ropinirole;

FIG. 10A graphically illustrate the effect of the permeation enhancing system of the present invention on the 24-hour cumulative drug permeation of anti-nausea drug granisetron;

FIG. 10B graphically illustrate the effect of the permeation enhancing system of the present invention on the drug instant flux of anti-nausea drug granisetron;

FIG. 11A graphically illustrate the effect of the permeation enhancing system of the present invention on the 24-hour cumulative drug permeation of anti-nausea drug ondansetron;

FIG. 11B graphically illustrate the effect of the permeation enhancing system of the present invention on the drug instant flux of anti-nausea drug ondansetron;

### DETAILED DESCRIPTION OF THE PREFERRED EMBODIMENTS

The invention now provides a formulation which shows adequate transdermal penetration enhancement effect for numerous therapeutic compounds belonging to different chemical groups. Accordingly, the present invention now provides a skin permeation enhancing system comprising a first component that is a polyalcohol, preferably propylene glycol; a second component that is a monoalkyl ether of diethylene glycol, preferably diethylene glycol monomethyl ether or diethylene glycol monoethyl ether; and preferably also a third component that is a saturated fatty alcohol or fatty acid given by the formula CH₃—(CH₂)ₙ—CH₂OH or CH₃—(CH₂)ₙ—H₂COOH, respectively, in which n is an integer from 8 to 22, preferably 8 to 12, most preferably 10 or an unsaturated fatty alcohol or fatty acid given by the formula CH₃—(CₙH₂₍ₙ₋ₓ₎)—OH or CH₃—(CₙH₂₍ₙ₋ₓ₎)—COOH, respectively, in which n is an integer from 8 to 22, in a solvent system comprising a C₁-C₄ alkanol, preferably ethanol; and purified water.

Optionally the composition may also comprise additional compounding aids such as, but not limited to, one or more thickening agents such as polyacrylic acids such as carbopol, cellulose derivatives such as hydroxypropylcellulose, povidone, poloxamers, alginates, gums, polyacrylamide/isoparaffin/laureth-7, polymethyl vinyl ether/maleic anhydride copolymers, pectins, and any thickening agent known in the art. The composition of the present invention may also further comprise other pharmaceutical compounding agents such as, but not limited to, pH regulators, preservatives, flavors, fragrances, sweeteners, stabilizers, anti-oxidants, chelatants, surfactants, solubilizers, humectants, film-forming agents, and the like.

Compositions of the present invention are any one of a variety of dosage forms for use in the continuous administration of systematically active drugs by absorption through the skin or the mucosa. Suitable dosage forms include creams, lotions, gels, ointments, mousses, sprays, aerosols, and patches. Preferred dosage forms are semi-solid gels. Even preferred dosage forms are non occlusive.

The transdermal delivery system of the present invention may comprise one or more active agents, or a mixture thereof. The term “drug” or “active drug” or “active agents” or “pharmaceutical active drug” or “therapeutic agent” or “pharmacologically active agent” as used herein to describe the principal active ingredient(s) of the composition intends a biologically active compound or a mixture of compounds that has a therapeutic, prophylactic or other beneficial pharmacological and/or physiological effect on the wearer of the composition.

Examples of types of drugs that may be used in the composition of the present invention include systemically active agents acting on the Central Nervous System (CNS). Suitable CNS drugs include drugs to treat Parkinson disease; drugs to treat Alzheimer disease and senile dementia; Attention Deficit and Hyperactivity Disorders (ADHD) drugs; drugs to treat narcolepsy; anti-anxiety drugs; anti-depression drugs; drugs to treat epilepsy; drugs to treat insomnia; drugs to treat motor neurone diseases, such as Huntington's chorea; drugs to treat multiple sclerosis; anti-nausea and anti-vomiting drugs; anti-psychotic drugs; hypnotics; anti-depressants; tranquilizers; drugs to treat Restless Legs Syndrome (RLS); drugs to treat addictive behaviors, such as alcohol addiction, nicotine addiction, drug addiction, food addiction; central analgesics; drugs to treat central metabolism disorders; etc.

Examples of anti-Parkinson drugs that may be used in the composition of the present invention include, but are not limited to, amantadine, benserazide, carbidopa, levodopa, benztropine, biperiden, benzhexol, procyclidine, bornaprine, budipine, entacapone, ethopropazine, lazabemide, memantine, orphenadrine, selegiline, tolcapone, trihexyphenidyl, modafinil, talampanel, altinicline, brasofensine, safinamide, droxidopa, rasagline, bromocriptine, cabergoline, pergolide, piribedil, pramipexole, quinagolide, terguride, rotigotine, riluzole, talipexole, piroheptine, bifeprunox, spheramine, lisuride, sumanirole, ropinirole, rotigotine, pharmaceutically acceptable derivatives such as salts and isomers thereof, pharmaceutically acceptable pro-drugs thereof, and mixtures thereof. Preferred anti-Parkinson agents are ropinirole, pramipexole, selegiline, lisuride, and pergolide.

Examples of anti-Alzheimer drugs that may be used in the composition of the present invention include, but are not limited to, choline esterase inhibitors such as tacrine, donepezil, rivastigmine, galantamine, amantadine, pharmaceutically acceptable derivatives such as salts and isomers thereof, pharmaceutically acceptable pro-drugs thereof, and mixtures thereof. Preferred anti-Alzheimer agents are donepezil and rivastigmine.

Examples of analgesics drugs that may be used in the composition of the present invention include, but are not limited to, alfentanil, buprenorphine, butorphanol, codeine, dextromoramide, dextropropoxyphene, dezocine, diamorphine, dihydrocodeine, fentanyl, flupirtine, hydrocodone, hydromorphone, ketobemidone, levomethadyl, mepiridine, meptazinol, methadone, morphine, nalbuphine, oxycodone, papaveretum, pentazocine, pethidine, phenoperidine, piritramide, remifentanil, tilidine, tramadol, sufentanil, pharmaceutically acceptable derivatives such as salts and isomers thereof, pharmaceutically acceptable pro-drugs thereof, and mixtures thereof. Preferred analgesics are buprenorphine and fentanyl.

Examples of anti-addiction drugs that may be used in the composition of the present invention include, but are not limited to, nicotine, buprenorphine, naloxone, pharmaceutically acceptable derivatives such as salts and isomers thereof, pharmaceutically acceptable pro-drugs thereof, and mixtures thereof.

Examples of anti-psychotic drugs that may be used in the composition of the present invention include, but are not limited to, phenothiazines such as chlorpromazine, fluphenazine, perphenazine, prochlorperazine, thioridazine, trifluoperazine; butyrophenones such haloperidol, droperidol, pimozide; clozapine, olanzapine, mirtanzapine, tinaeptine, bupropion, risperidone, quetiapine, ziprasidone, amisulpride, melperone, paliperidone, aripiprazole, pharmaceutically acceptable derivatives such as salts and isomers thereof, pharmaceutically acceptable pro-drugs thereof, and mixtures thereof.

Examples of anti-anxiety drugs that may be used in the composition of the present invention include, but are not limited to, benzodiazepines such as alprazolam, bromazepam, diazepam, lorazepam, clonazepam, temazepam, oxazepam, flunitrazepam, triazolam, chlordiazepoxide, flurazepam, estazolam, nitrazepam, pharmaceutically acceptable derivatives such as salts and isomers thereof, pharmaceutically acceptable pro-drugs thereof, and mixtures thereof.

Examples of anti-depressant drugs that may be used in the composition of the present invention include, but are not limited to, selective serotonine reuptake inhibitors (SSRIs) as citalopram, escitalopram oxalate, fluoxetine, fluvoxamine, paroxetine, sertraline, dapoxetine; serotonin-norepinephrine reuptake inhibitors (SNRIs) such as venlafaxine and duloxetine; monoamine oxidase inhibitors (MAOI) such as harmaline, iproniazid, isocarboxazid, nialamide, pargyline, phenelzine, selegiline, toloxatone, tranylcypromine, brofaromine, moclobemide; tricyclic anti-depressants such as amitriptyline, amoxapine, butriptyline, clomipramine, desipramine, dibenzepin, dothiepin, doxepin, imipramine, iprindole, lofepramine, melitracen, nortriptyline, opipramol, protriptyline, trimipramine; tetracyclic anti-depressants such as maprotiline, mianserin, nefazodone, trazodone; pharmaceutically acceptable derivatives such as salts and isomers thereof, pharmaceutically acceptable pro-drugs thereof, and mixtures thereof.

Examples of anti-insomnia drugs that may be used in the composition of the present invention include, but are not limited to, zolpidem, zopiclone, pharmaceutically acceptable derivatives such as salts and isomers thereof, pharmaceutically acceptable pro-drugs thereof, and mixtures thereof.

Examples of drugs treating ADHD that may be used in the composition of the present invention include, but are not limited to, methylphenidate, pharmaceutically acceptable derivatives such as salts and isomers thereof, pharmaceutically acceptable pro-drugs thereof, and mixtures thereof.

The present invention could be applied to other groups of pharmaceutical active agents for instance for alpha-adrenergic agonists such as budralazine, clonidine, epinephrine, fenoxazoline, naphazoline, phenylephrine, phenylpropanolamine, beta-adrenergic agonists such as formoterol, methoxyphenamine, alpha-adrenergic blockers such as doxazosin, prazosin, terazosin, trimazosin, yohimbine, beta-adrenergic blockers such as atenolol, bisoprolol, carteolol, carvedilol, metoprolol, nadolol, penbutolol; anti-neoplastic agents such as 5-fluorouracil, etc; anti-inflamatory agents; anesthetics; anti-androgens; anti-anginals; anti-cholinergics; anti-convulsants; anti-estrogen such as tamoxifen, 4-OH tamoxifen; anti-histaminics; bronchodilators; diuretics; glucocorticoids; muscle relaxants; narcotic antagonists; etc.

It is to be understood herein that the active agent is intended to mean a single active agent or a combination of more than one active agent.

The amount of the systemically and/or topically active agent included in the formulation is subject to the degree to which penetration enhancement is achieved. In the preferred embodiments, the active agents are CNS drugs such as CNS drugs present in the compositions at a concentration depending on the active agent and on the therapeutic effect desired. In the preferred embodiments, the permeation enhancer system comprises a first component that is a monoalkyl ether of diethylene glycol, preferably diethylene glycol monomethyl or diethylene glycol monoethyl ether in amount up to 30% w/w, preferably from about 0.2 to 15% w/w and more preferably 2.5 to 7.5% w/w; a second component that is a glycol such as propylene glycol, butylene glycol, hexylene glycol, ethylene glycol, preferably propylene glycol in about 0.5 to about 30% w/w; preferably from about 5 to 20% w/w and more preferably 5 to 15% w/w; and preferably also a third component that is a saturated fatty alcohol and/or fatty acid given by the formula CH₃—(CH₂)ₙ—CH₂OH or CH₃—(CH₂)ₙ—H₂COOH, respectively, in which n is an integer from 8 to 22, preferably 8 to 12, most preferably 10 or an unsaturated fatty alcohol and/or fatty acid given by the formula CH₃—(CₙH₂₍ₙ₋ₓ₎)—OH or CH₃—(CₙH₂₍ₙ₋ₓ₎)—COOH, respectively, in which n is an integer from 8 to 22. Preferred compositions in accordance with the present invention contain a fatty alcohol, preferably decyl or lauryl or myristyl in about 0. 1 to about 20% w/w on the whole composition; preferably from about 0.4 to 10% w/w, and more preferably 0.2 to 2% w/w. In the preferred embodiments, the binary vehicle composite comprises a C₂-C₄ alkanol and purified water.

The compositions in accordance with the present invention contain an alcohol such as ethanol, isopropanol, n-propanol, butanol, preferably ethanol in an amount of about 5 to about 75% w/w; preferably from about 15% to about 65% w/w and more preferably from about 20 to 55% w/w. In the preferred embodiments, the compositions in accordance with the present invention further comprise a gelling agent, e.g. carbomer, carboxyethylene or polyacrylic acid such as Carbopol 980 or 940 NF, 981 or 941 NF, 1382 or 1342 NF, 5984 or 934 NF, ETD 2020, 2050, 934P NF, 971P NF, 974P NF, Noveon AA-1 USP, etc; cellulose derivatives such as ethylcellulose, hydroxypropylmethylcellulose (HPMC), ethylhydroxyethylcellulose (EHEC), carboxymethylcellulose (CMC), hydroxypropylcellulose (HPC) (Klucel different grades), hydroxyethylcellulose (HEC) (Natrosol grades), HPMCP 55, Methocel grades, etc; natural gums such as arabic, xanthan, guar gums, alginates, etc; polyvinylpyrrolidone derivatives such as Kollidon grades; polyoxyethylene polyoxypropylene copolymers such as Lutrol F grades 68, 127, etc; others like chitosan, polyvinyl alcohols, pectins, veegun grades, etc. In the present invention, Lutrol F grades and Carbopol grades were preferred. Those of the skill in the art should know of other gelling agents that are suitable to practice the present invention. The gelling agent is present from about 0.2 to about 30% w/w depending on the type of polymer.

In the preferred embodiments, the compositions in accordance with the present invention further comprise a pH adjusting agent which can optionally have crosslinking function e.g. a ternary amine such as triethanolamine, tromethamine, tetrahydroxypropylethylendiamine, etc; NaOH solution, etc. The pH regulator is present in the formulations in about 0.05 to about 20% w/w. Other ingredients can optionally be included in compositions of the present invention, for example, preservatives and/or anti-oxidants such as sulfites, propyl gallate, DL-alfa tocoferol, chelatants, buthylhydroxytoluene, buthylhydroxyanisole, ethylenediaminetetraacetic acid and its sodium salts, etc; co-solvents or solubilizers such as glycerol, polyethylene glycols, polyethylene glycols derivatives, polyethyleneglycol 660 hydroxystearate (Solutol HS15 from BASF), buthylene glycol, hexylene glycol, etc.

As such, in another aspect, the present invention relates to a method for administering topically or systemically active agent(s), comprising: an active agent(s); a penetration enhancer system (composed by a diethylene glycol monoalkyl ether, a glycol, and preferably a fatty alcohol and/or a fatty acid; a binary vehicle composite (composed by a C₁-C₄ alkanol and water); a gelling agent and a pH regulator.

It has been discovered that in a transdermal formulation comprising different group of drugs as active agents; a mixture of diethylene glycol monoethyl ether, propylene glycol and decyl or lauryl or myristyl alcohol as penetration enhancing system, in a binary vehicle composite comprised of ethanol and purified water, using a polymer or copolymer of acrylic acid, preferably a carbomer, or a cellulose derivative as thickening agent provides therapeutically effective serum concentration of each active agent throughout at least a 24-hour period as it is concluded when a bioavailability study of the above mentioned formulations were carried out in human beings volunteers.

The main aim followed by the present invention is to rapidly create a high concentration of the drug(s) in contact with the skin or mucosa attained by the careful combination of permeation enhancers and vehicles.

It is well known by the ones skilled in the art that an additive or a synergistic effect could be expected when two or more penetration enhancers are combined and included into a formulation. However, it is by no mean obvious to obtain an adequate penetration enhancement factor and a sustained flux of the active agent(s), achieving therapeutic effective levels, also controlled and sustained, by only one daily application of the formulation.

Accordingly, we can postulate that the behavior of the formulation of the present invention is due to the addition of several phenomena especially on the stratum corneum.

Although the mechanism of such stratum corneum effect in the present invention is not fully clear by the scientific knowledge up to now, it can be understood as follows: 1) the diethylene glycol monoethyl ether dissolves both hydrophilic and lipophilic active agent(s) therein and facilitates the penetration of the active agents to the skin; 2) propylene glycol, a widespread pharmaceutical vehicle, acts as a cosolvent of the drugs hence increase the solubility of the active agent(s) in the formulation and solvates the intracellular keratin of the stratum corneum and thus enhances drug mobility and skin hydration; 3) the fatty alcohol and/or the fatty acid is mainly distributed to the stratum corneum because of its lipophilicity and interacts with the stratum corneum lipids; 4) an alkanol, such as ethanol, also has a function to increase the stratum corneum liquid fluidity or a function to extract lipids from the stratum corneum; 5) water serves to augment the solubility of a hydrophilic active agent in the formulation and accelerates the release of lipophilic active agent from a formulation of the present invention.

In the preferred embodiment of the present invention, the active agents and the compounds which enhance said active agents penetration rate (namely, diethylene glycol monoethyl ether, propylene glycol and also preferably a fatty alcohol and/or a fatty acid such as decyl, lauryl or myristyl alcohol) are dissolved in a binary vehicle composite of an alkanol having C₁-C₄ atoms, preferably ethanol, and purified water.

The present invention relates to a novel composition for transdermal or transmucosal application of active agents to humans in an optimized dosage form and methods for providing therefrom a controlled and sustained administration of different group of said drugs.

The present invention also demonstrates its applicability not only for CNS drugs, but also for different groups of pharmaceutical active agents as disclosed herein.

### DEFINITION OF TERMS

“Penetration enhancement” or “permeation enhancement” as used herein relates to an increase in the permeability of skin to a pharmacologically active agent, i.e., so as to increase the rate at which the drug permeates through the skin and enters the bloodstream. The enhanced permeation effected through the use of such enhancers, and in particular, through the use of the enhancer composition of the present invention, can be observed by measuring the rate of diffusion of drug through animal or human skin using a diffusion cell apparatus as described in the examples herein.

An “effective” or an “adequate” permeation enhancer as used herein means a permeation enhancer that will provide the desired increase in skin permeability and correspondingly, the desired depth of penetration, rate of administration, and amount of drug delivered.

By “transdermal” delivery, applicants intend to include both transdermal (or “percutaneous”) and transmucosal administration, i.e., delivery by passage of a drug through the skin or mucosal tissue and into the bloodstream.

“Carriers” or “vehicles” as used herein refer to carrier materials suitable for transdermal drug administration, and include any such materials known in the art, e.g., any liquid, gel, solvent, liquid diluent, solubilizer, or the like, which is non toxic and which does not interact with other components of the composition in a deleterious manner. Examples of suitable vehicles for use herein include water, alcohols, polyalcohols, and glycols.

By the term “pharmacologically active agent” or “drug” as used herein is meant any chemical material or compound suitable for transdermal or transmucosal administration which induces a desired systemic effect.

By “controlled” is meant reduce or minimize plasmatic peaks and troughs normally present in some routes of administration, such as the oral route, of a pharmacologically active agent.

By “sustained” is meant extended maintenance of steady state plasma levels.

By “therapeutically effective” amount of a pharmacologically active agent is meant sufficient amount of a compound to provide the desired therapeutic effect, avoiding high or low plasmatic levels, obtaining, therefore, plasmatic levels of active within the therapeutic window.

### EXAMPLES

In order to further illustrate the present invention and the advantages thereof, the following specific examples are given. It is being understood that the examples herein disclosed are intended only as illustrative and in nowise limitative. Although pramipexole, ropinirole, rivastigmine, fentanyl, selegiline, pergolide, granisetron, and ondansetron are used herein the following examples as drug models illustrating the present invention, it will be appreciated by those skilled in the art that many other CNS drugs can similarly be used.

All the examples were prepared basically as follows: an alcoholic phase (solution containing the organo-soluble active drugs, the fatty alcohol, diethylene glycol monoethyl ether (Transcutol P), propylene glycol and ethanol, or some of them according to the formulation) was prepared. Water (and hydrosoluble active drugs) was then added and mixed to the organic solution. The thickening agent (carbomer or cellulose) was then added to the hydro-alcoholic active solution. pH-adjusting agent, if any, was finally added.

In the following examples, evaluation of formulations containing an anti-Parkinson drug, an anti-Alzheimer drug, an analgesic drug, or an anti-nausea drug was performed using a predictive experimental in vitro permeation model. Pre-clinical in vitro testing of transdermal formulations of the present invention was performed using static vertical diffusion cells, which simulates the physiological conditions of in vivo. The model consists of two compartments, donor and receptor, separated by a model skin membrane. The drug formulation is applied onto the skin surface which is maintained at a physiological temperature, and the permeated drug is collected in the receptor compartment containing a physiological receptor medium at regular intervals. Sample HPLC analysis shows a drug kinetic profile, with cumulative absorbed amount as a function of time, as well as a drug flux profile, which is the slope of the former as a function of time, and therefore allows characterization of release properties of the formulations.

The study was performed according the Organisation for Economic Cooperation and Development (OECD) guidance, “Guidance document for the conduct of skin absorption studies” (Mar. 5, 2004). The following conditions were implemented.

1. Diffusion cells: Vertical glass Franz diffusion cells with a receptor compartment of 7.5±0.3 mL and a donor compartment of 3 mL and a diffusion area of 1.77 cm² (see Table 1).

TABLE 1
Diffusion Cell Specification

| Type of cells | Franz vertical | |
|---|---|---|
| Diffusion area | 1.77 | cm² |
| Donor compartment volume | 3 | mL |
| Receptor compartment volume | 7.5 ± 0.3 | mL |

2. Receptor solution: Phosphate buffered saline (PBS) at pH 7.4, with addition of 2% w/v Volpo N20 (Oleth-20, oleyl ether of polyoxyethylene glycol), maintained at 35° C. during the whole study and stirred at 600 RPM (see Table 2).

TABLE 2
Properties of Receptor Solution

| Receptor medium | PBS + Volpo N20 2% |
|---|---|
| Receptor temperature | 35° C. |
| Receptor stirring speed | 600 RPM |

3. Formulation loading: About 10 mg (5.6 mg/cm²) of the formulation was applied with the tip of a plastic syringe plunger and gently spread over the skin diffusion surface. This loading is close to clinical loading, and is consistent with OECD guidelines. Formulations were left in non-occluded conditions, in order to allow the formulations to change as under in-use conditions.

4. Replicates: Overall, twelve skin samples from 3 different donors were used in randomized order over three different donors, one of which was used as internal reference.

5. Excised skin: Fresh pig ear was harvested and processed the same day of permeation (maximum 5 hours delay). The skin samples were sliced, and the thickness of each skin sample was measured with a micrometer. The samples were pre-incubated (for stabilization) for 2 hours on the receptor compartment, in contact with the receptor solution. The porcine skin has been found to have similar morphological and functional characteristics as human skin (see Simon G. A., Maibach H. I., “The pig as an experimental animal model of percutaneous permeation in man: qualitative and quanti-tative observations,” Skin Pharmacol. Appl. Skin Physiol., 13(5):229-34 (September-October 2000)). In addition, it has been found to permeability characteristics close to that of the human skin (see Andega S., Kanikkannan N., Singh M., “Comparison of the effect of fatty alcohols on the permeation of melatonin between porcine and human skin,” J. Control Release 77(1-2):17-25 (November 2001); Singh S., Zhao K., Singh J., “In vitro permeability and binding of hydrocarbons in pig ear and human abdominal skin,” Drug Chem. Toxicol. 25(1):83-92 (February 2002); Schmook F. P., Meingassner J. G., Billich A., “Comparison of human skin or epidermis models with human and animal skin in in-vitro percutaneous absorption,” Int. J. Pharm. 215(1-2):51-6 (March 2001)). Pig ear skin was preferred over human skin because of its greater supply.

TABLE 3
Properties of Skin Model

| | |
|---|---|
| Species | Pig |
| Gender | Male/Female |
| Age | 5-6 months |
| Region | Ear |
| Origin | Cadaver |
| Condition | Fresh |
| Time harvest/use | Max. 5 h |
| Pre-treatment | None |
| Membrane thickness | Sliced, 1000 ± 200 μm |

6. Skin integrity: Skin integrity was assessed by evaporimetry (TEWL). Skin samples with TEWL>50 g/cm²h were discarded and replaced.

7. Study duration: 24 hours, to correspond to formulations designed to be applied once daily.

8. Sampling frequency: variable.

9. Sampling: The studies were performed with a Microette® autosampler (Hanson Research). Receptor solution samples (1.2 mL) were automatically removed at regular interval times (after 0.8 mL receptor compartment priming). Samples were collected in 2 mL HPLC amber glass vials pre-sealed with septum crimp-caps and already containing 10 μL of a solution of 10% trifluoroacetic acid (TFA), for precipitation of macromolecules such as proteins released from the skin (see Table 4).

TABLE 4
Properties of Sampling Performed

| Type of sampling | | Automatic |
|---|---|---|
| Sample volume | [mL] | 1.2 |
| Waste volume | [mL] | 0.8 |

10. Samples processing: Samples were first transferred into Eppendorf microtubes, and centrifuged at 14500 RPM for 10 minutes. Each supernatant (0.9 mL) was then transferred in a clean 2 mL HPLC amber glass vial and crimp-capped, ready for analysis.

11. Sample analysis: Analysis of the samples was performed by HPLC with UV diode-array detection.

### Example A

### Transdermal Delivery of an Anti-Parkinson Drug (Pramipexole)

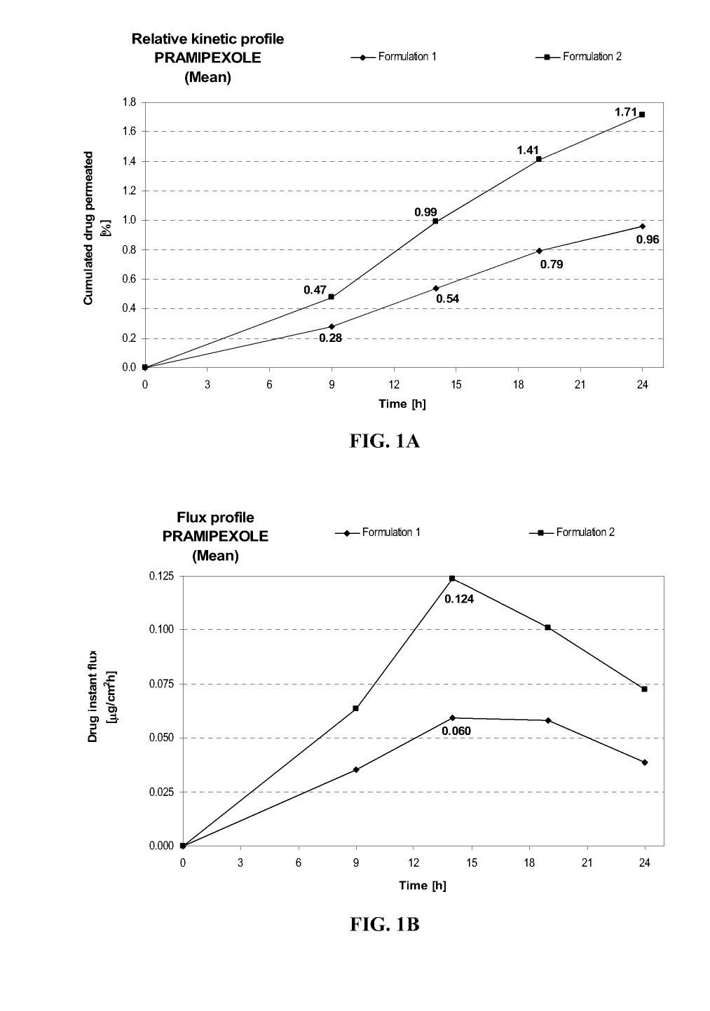

Formulation 2 delivers after 24 hours at similar pH (8.2±0.1) about 1.8 times more pramipexole than Formulation 1, as shown in FIG. 1A. This comparison shows the importance of the combination of diethylene glycol monoethyl ether and propylene glycol on the transdermal delivery of pramipexole. Similarly, the maximum instant pramipexole flux was about 2.1 times higher for Formulation 2 than for Formulation 1, as shown in FIG. 1B.

| | FORMULATION | |
|---|---|---|
| | Formulation 1 | Formulation 2 |
| Composition | % w/w | % w/w |
| Pramipexole dihydrochloride | 2.00 | 2.00 |
| (as free base equivalent) | | |
| Diethylene glycol monoethyl ether | — | 5.00 |
| Propylene glycol | — | 15.0 |
| Hydroxypropylcellulose | 1.50 | 1.50 |
| Ethanol, absolute | 40.0 | 40.0 |
| Sodium hydroxide | qs pH 8.2 +/ 0.1 | qs pH 8.2 +/ 0.1 |
| Purified water | qs 100.00 | qs 100.00 |

### Example B

### Transdermal Delivery of an Anti-Parkinson Drug (Pramipexole)

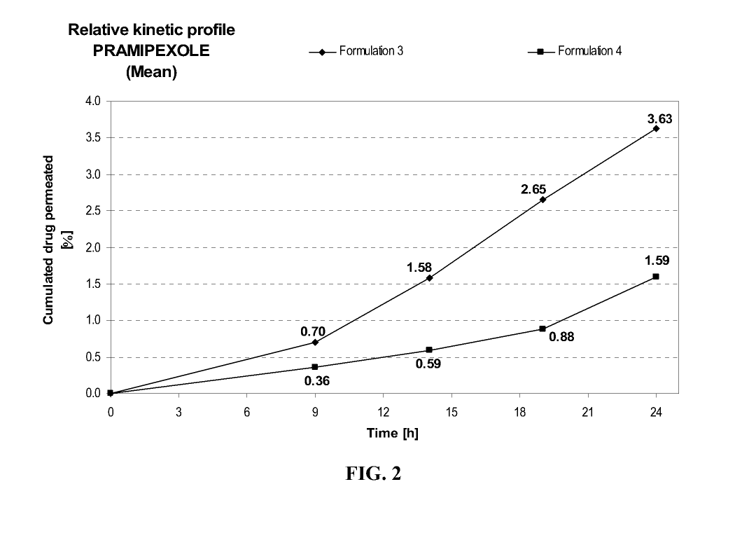

Formulation 4 delivers after 24 hours at similar pH (7.7±0.1) about 2.3 times more pramipexole than Formulation 3, as shown in FIG. 2. This comparison shows the importance of further adding a fatty compound such as myristyl alcohol to the combination of diethylene glycol monoethyl ether and propylene glycol on the transdermal delivery of pramipexole.

| | FORMULATION | |
|---|---|---|
| | Formulation 3 | Formulation 4 |
| Composition | % w/w | % w/w |
| Pramipexole dihydrochloride | 2.00 | 2.00 |
| (as free base equivalent) | | |
| Diethylene glycol monoethyl ether | 5.00 | 5.00 |
| Propylene glycol | 20.0 | 20.0 |
| Myristyl alcohol | — | 1 |
| Hydroxypropylcellulose | 1.50 | 1.50 |
| Anti-oxidant | 0.40 | 0.40 |
| Ethanol, absolute | 40.0 | 40.0 |
| Triethanolamine | qs pH 7.7 +/ 0.1 | qs pH 7.7 +/ 0.1 |
| Purified water | qs 100.00 | qs 100.00 |

### Example C

### Transdermal Delivery of an Anti-Alzheimer Drug (Rivastigmine)

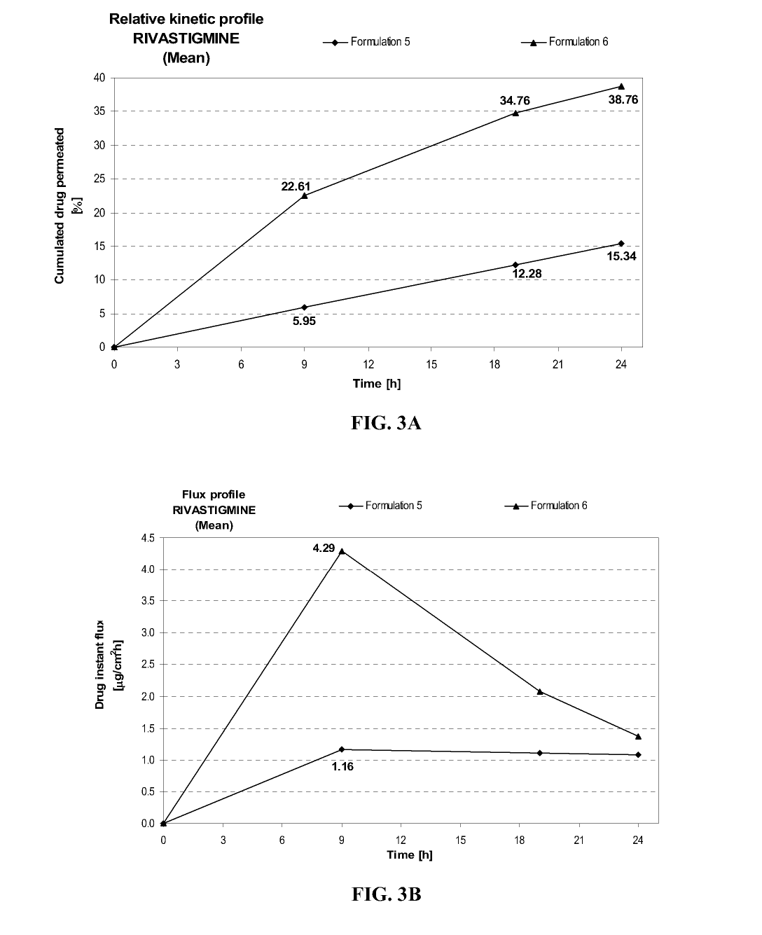

Formulation 6 delivers after 24 hours at similar pH (7.5±0.1) about 2.5 times more rivastigmine than Formulation 5, as shown in FIG. 3A. This comparison shows the importance of adding a permeation enhancing system composed of a fatty compound (myristyl alcohol), diethylene glycol monoethyl ether and propylene glycol, on the transdermal delivery of rivastigmine. Similarly, the maximum instant rivastigmine flux was about 3.7 times higher for Formulation 6 than for Formulation 5, as shown in FIG. 3B.

| | FORMULATION | |
|---|---|---|
| | Formulation 5 | Formulation 6 |
| Composition | % w/w | % w/w |
| Rivastigmine | 3.00 | 3.00 |
| (as free base equivalent) | | |
| Diethylene glycol monoethyl ether | — | 5.00 |
| Propylene glycol | — | 20.0 |
| Myristyl alcohol | — | 1 |
| Hydroxypropylcellulose | 1.50 | 1.50 |
| Ethanol, absolute | 45.0 | 45.0 |
| Triethanolamine | qs pH 7.5 +/ 0.1 | qs pH 7.5 +/ 0.1 |
| Purified water | qs 100.00 | qs 100.00 |

### Example D

### Transdermal Delivery of an Analgesic Drug (Fentanyl)

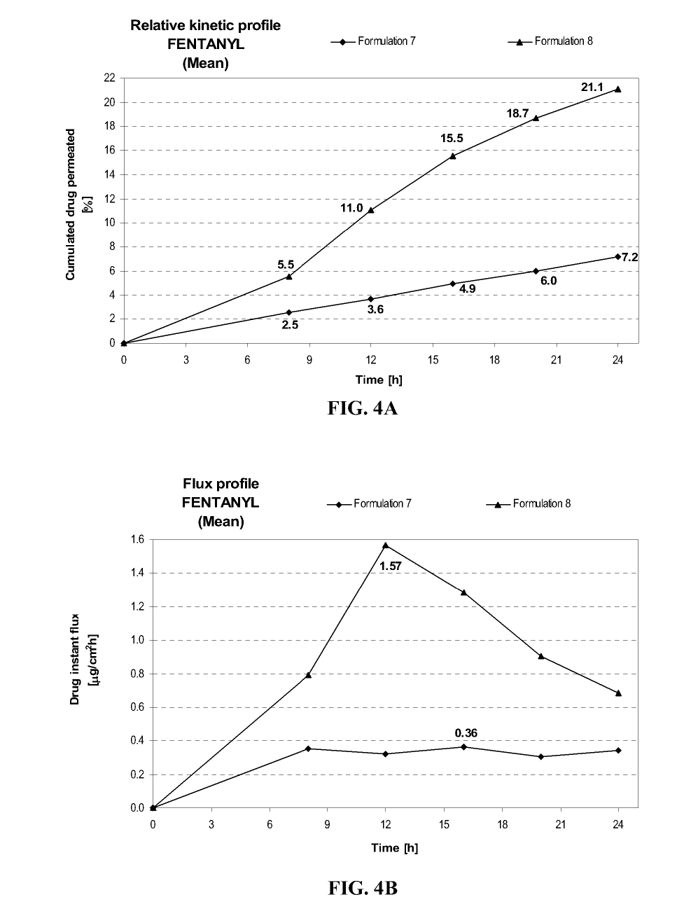

Formulation 8 delivers after 24 hours about 3 times more fentanyl than Formulation 7, as shown in FIG. 4A. This comparison shows the importance of the combination of diethylene glycol monoethyl ether and propylene glycol on the transdermal delivery of fentanyl. Similarly, the maximum instant fentanyl flux was about 4.4 times higher for Formulation 8 than for Formulation 7, as shown in FIG. 4B.

| | FORMULATION | |
|---|---|---|
| | Formulation 7 | Formulation 8 |
| Composition | % w/w | % w/w |
| Fentanyl (as free base equivalent) | 1.00 | 1.00 |
| Diethylene glycol monoethyl ether | — | 2.50 |
| Propylene glycol | — | 20.0 |
| Hydroxypropylcellulose | 1.50 | 1.50 |
| Ethanol, absolute | 40.0 | 40.0 |
| Purified water | qs 100.00 | qs 100.00 |

### Example E

### Transdermal Delivery of an Analgesic Drug (Fentanyl)

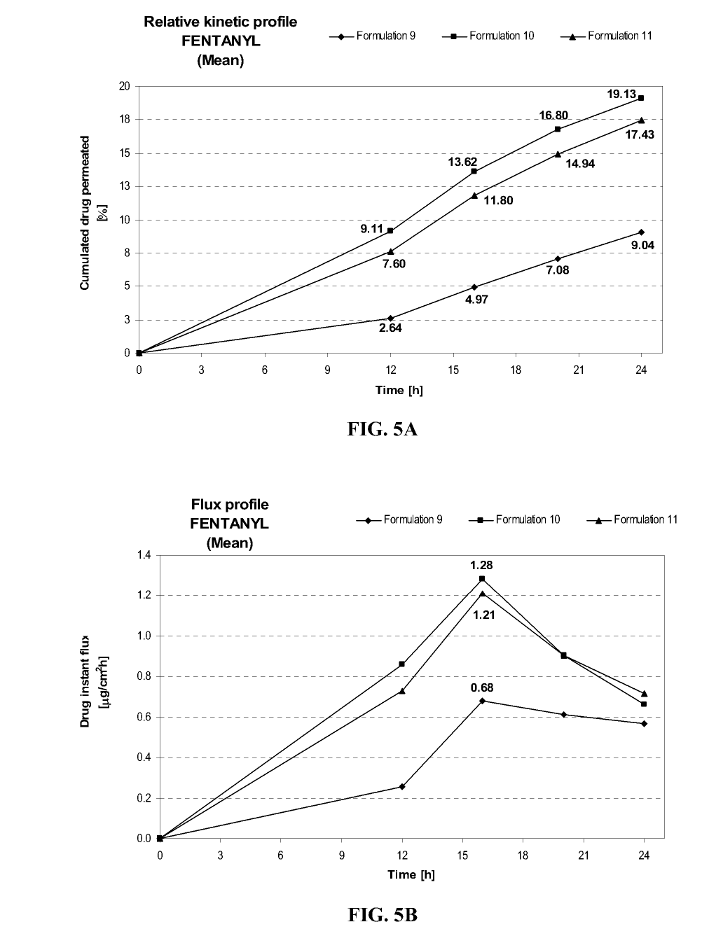

Formulation 10 and 11 deliver after 24 hours about 2 times more fentanyl than Formulation 9, as shown in FIG. 5A. This comparison shows the importance of further adding a fatty compound (lauryl alcohol or myristyl alcohol) to the combination of diethylene glycol monoethyl ether and propylene glycol, on the transdermal delivery of fentanyl. Similarly, the maximum instant fentanyl flux was about 1.8 times higher for Formulation 10 and Formulation 11 than for Formulation 9, as shown in FIG. 5B.

| | FORMULATION | | |
|---|---|---|---|
| | Formulation 9 | Formulation 10 | Formulation 11 |
| Composition | % w/w | % w/w | % w/w |
| Fentanyl (as free | 1.00 | 1.00 | 1.00 |
| base equivalent) | | | |
| Diethylene glycol | 2.50 | 2.50 | 2.50 |
| monoethyl ether | | | |
| Propylene glycol | 20.0 | 20.0 | 20.0 |
| Lauryl alcohol | — | 1.00 | — |
| Myristyl alcohol | — | — | 1.00 |
| Hydroxypropyl- | 1.50 | 1.50 | 1.50 |
| cellulose | | | |
| Ethanol, absolute | 40.0 | 40.0 | 40.0 |
| Purified water | qs 100.00 | qs 100.00 | qs 100.00 |

### Example F

### Transdermal Delivery of an Anti-Parkinson Drug (Selegiline)

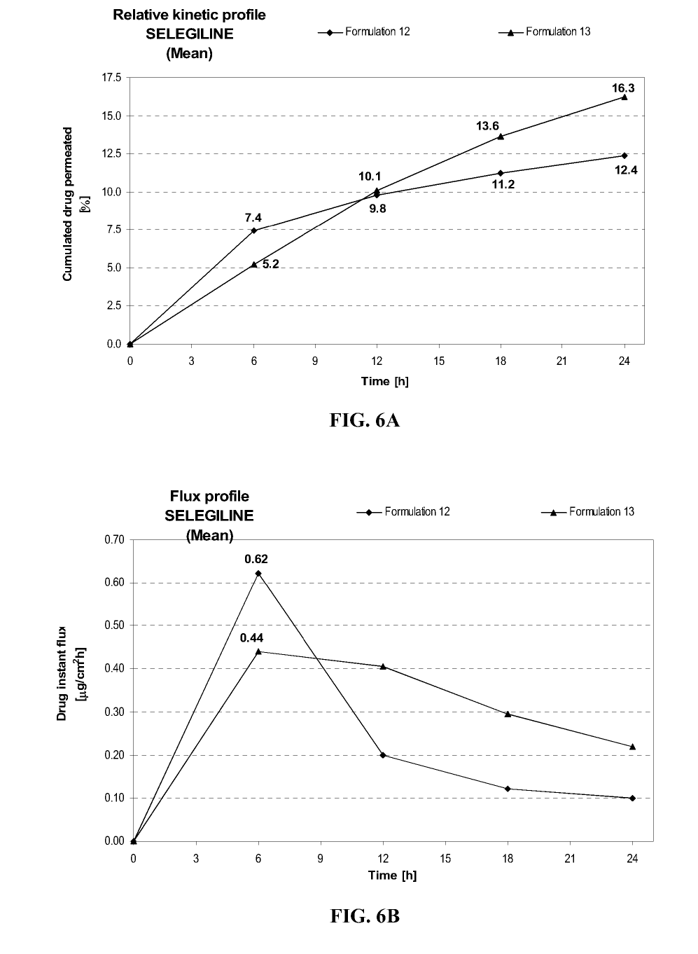

Formulation 13 delivers after 24 hours about 30 percent more selegiline than Formulation 12, as shown in FIG. 6A. This comparison shows the importance of adding a permeation enhancing system composed of a fatty compound (lauryl alcohol), diethylene glycol monoethyl ether and propylene glycol, on the transdermal delivery of selegiline. Maximum instant selegiline flux for Formulation 13 was about 70% those of Formulation 12, as shown in FIG. 6B. Noteworthy, drug flux is more sustained for Formulation 13 than for Formulation 12, where a steep drop is noticed after 6 hours. Sustained transdermal flux is an essential feature of the invention of the present invention, as it avoids troughs and peaks of plasmatic levels, often responsible for side effects and for lack of therapeutic effect.

| | FORMULATION | |
|---|---|---|
| | Formulation 12 | Formulation 13 |
| Composition | % w/w | % w/w |
| Selegiline hydrochloride | 1.00 | 1.00 |
| Diethylene glycol monoethyl ether | — | 5.00 |
| Propylene glycol | — | 10.0 |
| Lauryl alcohol | — | 1 |
| Hydroxypropylcellulose | 1.50 | 1.50 |
| Ethanol, absolute | 40.0 | 40.0 |
| Triethanolamine | 0.50 | 0.50 |
| Purified water | qs 100.00 | qs 100.00 |

### Example G

### Transdermal Delivery of an Anti-Parkinson Drug (Pergolide)

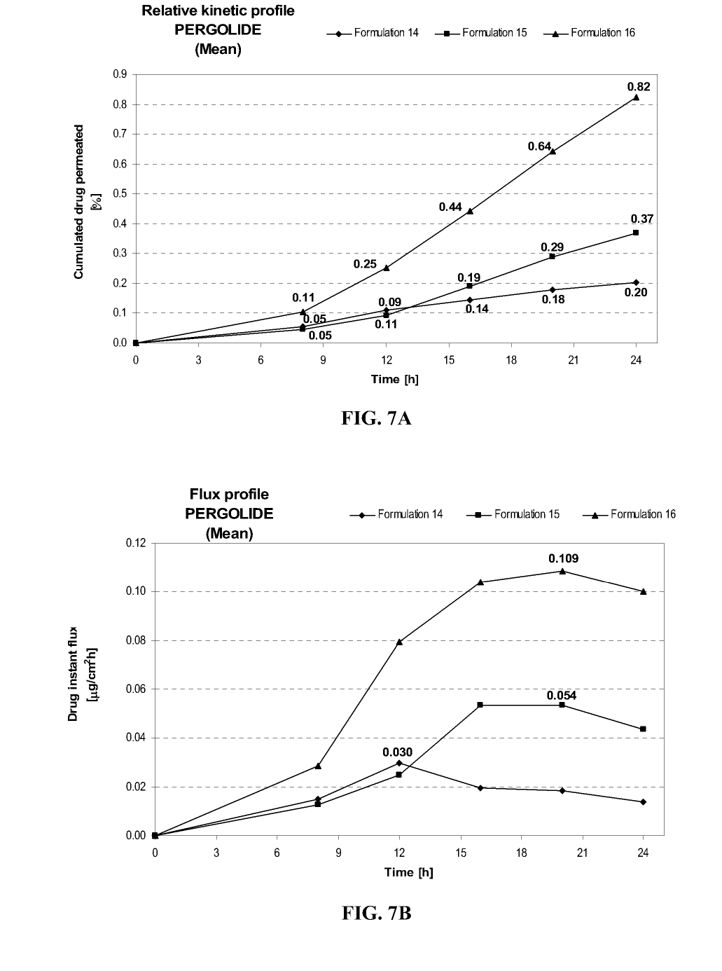

Formulation 15 delivers after 24 hours about 2 times more pergolide than Formulation 14, as shown in FIG. 7A. This shows the importance of adding a combination of diethylene glycol monoethyl ether and propylene glycol on the transdermal delivery of pergolide. Further adding a fatty compound (myristyl alcohol) to the combination of diethylene glycol monoethyl ether and propylene glycol (Formulation 14) results in about 2.2 even higher transdermal delivery of pergolide, i.e. in levels more than 4 times higher than for the reference formulation 14. Same conclusions are also valid when observing respective maximum instant pergolide flux for the three formulations, as shown in FIG. 7B.

| | FORMULATION | | |
|---|---|---|---|
| | Formulation | | |
| | 14 | Formulation 15 | Formulation 16 |
| Composition | % w/w | % w/w | % w/w |
| Pergolide mesylate | 1.00 | 1.00 | 1.00 |
| Diethylene glycol | — | 5.00 | 5.00 |
| monoethyl ether | | | |
| Propylene glycol | — | 10.0 | 10.0 |
| Myristyl alcohol | — | — | 1.00 |
| Hydroxypropylcellulose | 1.50 | 1.50 | 1.50 |
| Ethanol, absolute | 40.0 | 40.0 | 40.0 |
| Purified water | qs 100.00 | qs 100.00 | qs 100.00 |

### Example H

### Transdermal Delivery of an Anti-Parkinson Drug (Ropinirole)

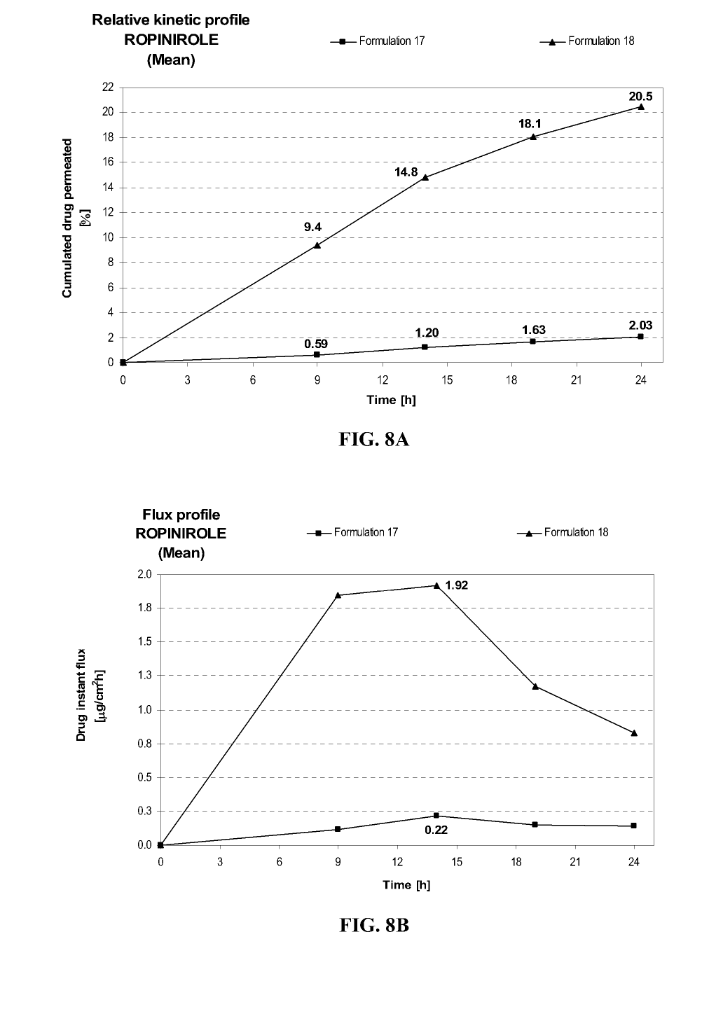

Formulation 18 delivers after 24 hours about 10 times more ropinirole than Formulation 17, as shown in FIG. 8A. This comparison shows the importance of adding a permeation enhancing system composed of a fatty compound (myristyl alcohol), diethylene glycol monoethyl ether and propylene glycol, on the transdermal delivery of ropinirole. Similarly, the maximum instant ropinirole flux for Formulation 18 was about 9 times higher than those of Formulation 17, as shown in FIG. 8B.

| | FORMULATION | |
|---|---|---|
| | Formulation 17 | Formulation 18 |
| Composition | % w/w | % w/w |
| Ropinirole | 3.00 | 3.00 |
| Diethylene glycol monoethyl ether | — | 5.00 |
| Propylene glycol | — | 20.0 |
| Myristyl alcohol | — | 1.00 |
| Hydroxypropylcellulose | 1.50 | 1.50 |
| Ethanol, absolute | 45.0 | 45.0 |
| HCl 1M | qs pH 7.6 +/0.3 | qs pH 7.6 +/0.3 |
| Purified water | qs 100.00 | qs 100.00 |

### Example I

### Transdermal Delivery of an Anti-Parkinson Drug (Ropinirole)

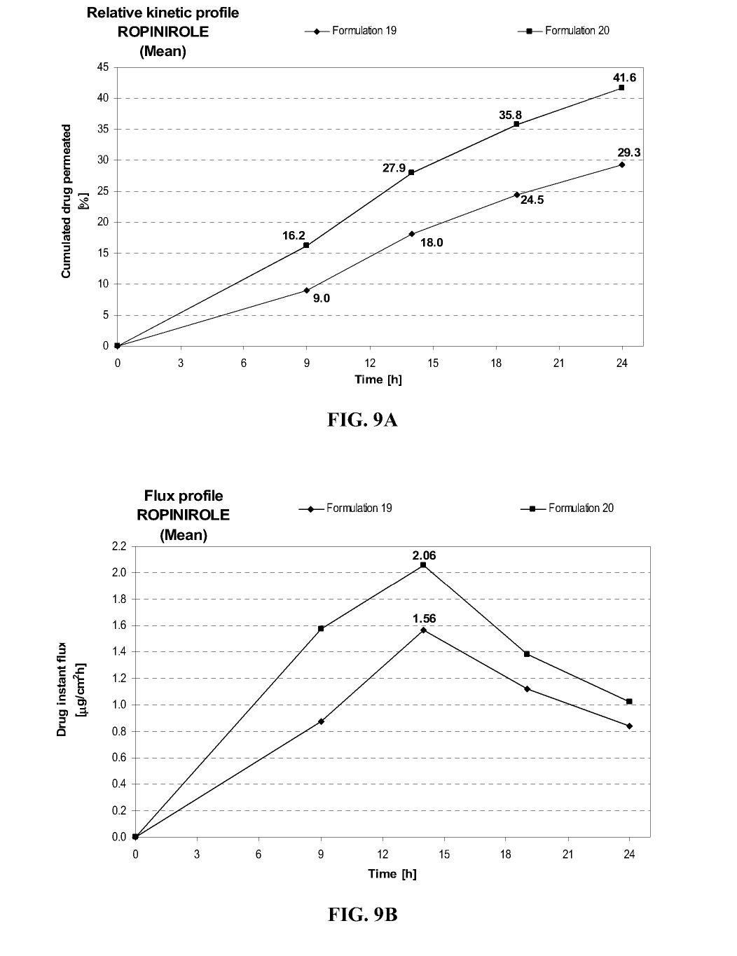

Formulation 20 delivers after 24 hours about 40 percent more ropinirole than Formulation 19, as shown in FIG. 9A. This comparison shows the importance of further adding a fatty compound (myristyl alcohol) to a combination of diethylene glycol monoethyl ether and propylene glycol, on the transdermal delivery of ropinirole. Similarly, the maximum instant ropinirole flux for Formulation 20 was about 30 percent higher than those of Formulation 19, as shown in FIG. 9B.

| | FORMULATION | |
|---|---|---|
| | Formulation 19 | Formulation 20 |
| Composition | % w/w | % w/w |
| Ropinirole | 1.50 | 1.50 |
| (as free base equivalent) | | |
| Diethylene glycol monoethyl ether | 5.00 | 5.00 |
| Propylene glycol | 20.0 | 20.0 |
| Myristyl alcohol | — | 1.00 |
| Anti-oxidant | 0.40 | 0.40 |
| Hydroxypropylcellulose | 1.50 | 1.50 |
| Ethanol, absolute | 45.0 | 45.0 |
| Triethanolamine | qs pH 8.1 +/0.1 | qs pH 8.1 +/0.1 |
| Purified water | qs 100.00 | qs 100.00 |

### Example J

### Transdermal Delivery of an Anti-Nausea Drug (Granisetron)

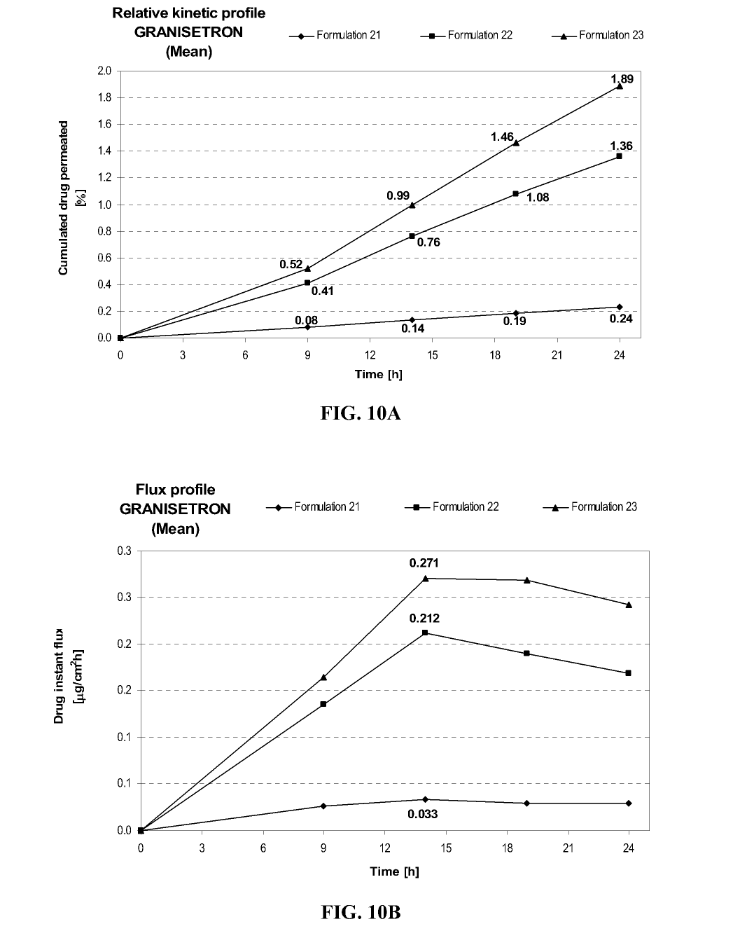

Formulation 22 delivers after 24 hours about 6 times more granisetron than Formulation 21, as shown in FIG. 10A. This shows the importance of adding a combination of diethylene glycol monoethyl ether and propylene glycol on the transdermal delivery of granisetron. Further adding a fatty compound (myristyl alcohol) to the combination of diethylene glycol monoethyl ether and propylene glycol (Formulation 23) results in about 40 percent even higher transdermal delivery of granisetron, i.e. in levels more than about 8 times higher than for the reference formulation 21. Same conclusions are also valid when observing respective maximum instant pergolide flux for the three formulations, as shown in FIG. 10B.

| | FORMULATION | | |
|---|---|---|---|
| | Formulation | | |
| | 21 | Formulation 22 | Formulation 23 |
| Composition | % w/w | % w/w | % w/w |
| Granisetron (as free | 1.00 | 1.00 | 1.00 |
| base equivalent) | | | |
| Diethylene glycol | — | 5.00 | 5.00 |
| monoethyl ether | | | |
| Propylene glycol | — | 20.0 | 20.0 |
| Myristyl alcohol | — | — | 1.00 |
| Hydroxypropylcellulose | 1.50 | 1.50 | 1.50 |
| Ethanol, absolute | 40.0 | 40.0 | 40.0 |
| Buffer, pH 4.00 | qs 100.00 | qs 100.00 | qs 100.00 |

### Example K

### Transdermal Delivery of an Anti-Nausea Drug (Ondansetron)

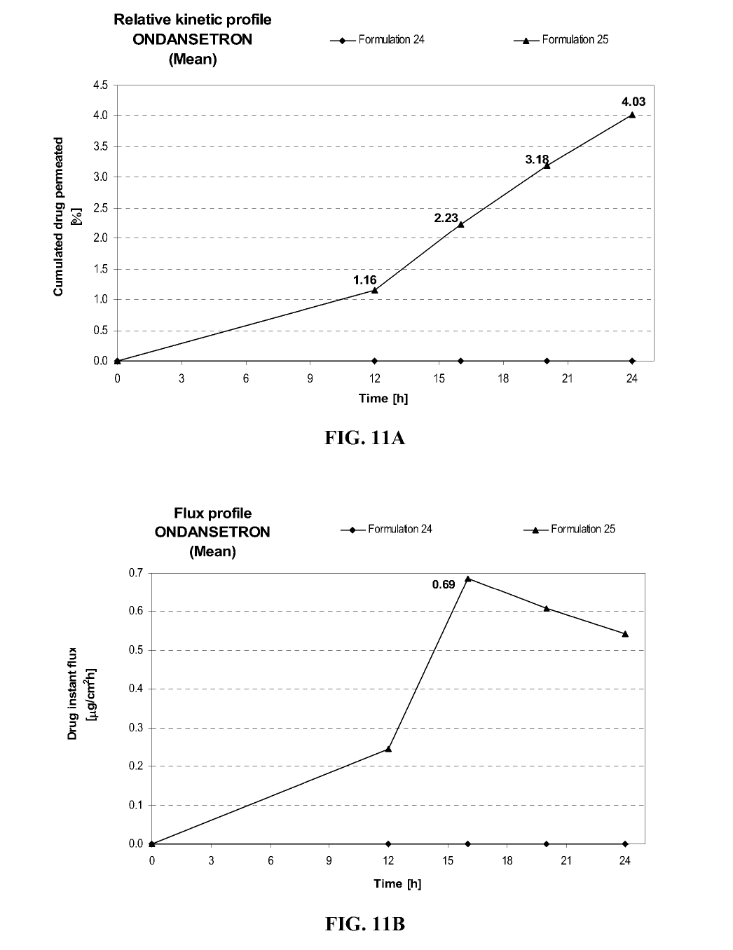

Formulation 25 delivers after 24 hours about 4.00% of ondansetron applied on the skin, albeit ondansetron levels delivered by Formulation 24 are so low that they are not even detectable by HPLC ananlysis, as shown in FIG. 11A. This comparison shows the importance of adding a permeation enhancing system composed of a fatty compound (myristyl alcohol), diethylene glycol monoethyl ether and propylene glycol, to ensure transdermal delivery of ondansetron at adequate therapeutic levels. Similarly, the maximum instant ondansetron flux reached about 0.7 mg/cm².h for Formulation 25, as shown in FIG. 11B.

| | FORMULATION | |
|---|---|---|
| | Formulation 24 | Formulation 25 |
| Composition | % w/w | % w/w |
| Ondansetron (as free base equivalent) | 1.00 | 1.00 |
| Diethylene glycol monoethyl ether | — | 5.00 |
| Propylene glycol | — | 10.0 |
| Myristyl alcohol | — | 1.00 |
| Hydroxypropylcellulose | 1.50 | 1.50 |
| Ethanol, absolute | 40.0 | 40.0 |
| Purified water | qs 100.00 | qs 100.00 |

What is claimed is:

1. A transdermal or transmucosal non-occlusive, semi-solid pharmaceutical formulation comprising:
at least one systemically active agent; and
a permeation enhancing solvent system present in an amount sufficient to solubilize the at least one active ingredient and characterized in that it includes:
(i) a pharmaceutically acceptable monoalkyl ether of diethylene glycol present in an amount of between about 1% and 30% by weight of the solvent system;
(ii) a pharmaceutically acceptable glycol present in an amount of between about 1% and 30% by weight of the solvent system, wherein the monoalkyl ether of diethylene glycol and the glycol in combination are present in an amount of at least 15% and no more than 60% by weight of the formulation; and
(iii) a mixture of a C₂ to C₄ alcohol and water, which mixture is present in an amount of between about 40% and 98% by weight of the solvent system, wherein the C₂ to C₄ alcohol is present in an amount of about 5% to 80% by weight of the mixture, and the water is present in an amount of about 20% to 95% by weight of the mixture; so that, compared to formulations not containing the present permeation enhancing solvent system, the present formulation (a) inhibits crystallization of the at least one active ingredient on a skin or mucosal surface of a mammal, (b) reduces or prevents transfer of the formulation to clothing or to another being, (c) modulates biodistribution of the at least one active agent within different layers of skin, (d) facilitates absorption of the at least one active agent by a skin or a mucosal surface of a mammal, or (e) provides a combination of one or more of (a) through (d).

2. The pharmaceutical formulation of claim 1, wherein the monoalkyl ether of diethylene glycol and the glycol are present in a weight ratio of 10:1 to 1:10.

3. The pharmaceutical formulation of claim 1, wherein the monoalkyl ether of diethylene glycol is selected from the group consisting of diethylene glycol monomethyl ether, and diethylene glycol monoethyl ether or mixtures thereof.

4. The pharmaceutical formulation of claim 1, wherein the glycol is selected from the group consisting of propylene glycol, dipropylene glycol or mixtures thereof.

5. The pharmaceutical formulation of claim 1, wherein the C₂ to C₄ alcohol is selected from the group consisting of ethanol, propanol, isopropanol, 1-butanol, 2-butanol, or mixtures thereof.

6. The pharmaceutical formulation of claim 1, wherein the formulation further includes a saturated fatty alcohol or fatty acid, or mixtures thereof, wherein said fatty alcohol and/or said fatty acid have the formula CH₃—(CH₂)ₙ—CH₂OH or CH₃—(CH₂)ₙ—H₂COOH, respectively, in which n is an integer from 8 to 22, preferably 8 to 12, most preferably 10; or an unsaturated fatty alcohol or fatty acid, or mixtures thereof, wherein said unsaturated fatty alcohol and/or fatty acid have the formula CH₃—(CₙH₂₍ₙ₋ₓ₎)—OH or CH₃—(CₙH₂₍ₙ₋ₓ₎)—COOH, respectively, in which n is an integer from 8 to 22.

7. The pharmaceutical formulation of claim 1, wherein the formulation further includes lauryl alcohol or myristyl alcohol present in an amount from 0.5 to 2% by weight of the total formulation.

8. The pharmaceutical formulation of claim 1, wherein the at least one systemically active agent is a drug that acts on the Central Nervous System (CNS) of a mammal.

9. The pharmaceutical formulation of claim 8, wherein the at least one systemically active agent is a drug to treat Parkinson disease; drugs to treat Alzheimer disease and senile dementia; Attention Deficit and Hyperactivity Disorders (ADHD) drugs; drugs to treat narcolepsy; anti-anxiety drugs; anti-depression drugs; drugs to treat epilepsy; drugs to treat insomnia; drugs to treat motor neurone diseases; drugs to treat multiple sclerosis; anti-nausea and anti-vomiting drugs; anti-psychotic drugs; hypnotics; anti-depressants; tranquilizers; drugs to treat Restless Legs Syndrome (RLS); drugs to treat addictive behaviors, such as alcohol addiction, nicotine addiction, drug addiction, food addiction; central analgesics; drugs to treat central metabolism disorders; or a combination of one of the previously mentioned drugs with another drug.

10. The pharmaceutical formulation of claim 9, wherein the at least one systemically active agent is an anti-Parkinson drug selected from the group consisting of amantadine, benserazide, carbidopa, levodopa, benztropine, biperiden, benzhexol, procyclidine, bomaprine, budipine, entacapone, ethopropazine, lazabemide, memanti-ne, orphenadrine, selegiline, tolcapone, trihexyphenidyl, modafinil, talampanel, altinicline, brasofensine, safinamide, droxidopa, rasagline, bromocriptine, cabergoline, pergolide, piribedil, pramipexole, quinagolide, terguride, rotigotine, riluzole, talipexole, piroheptine, bifeprunox, spheramine, lisuride, sumanirole, ropinirole, rotigotine, pharmaceutically acceptable salts, isomers and pro-drugs thereof, and mixtures thereof.

11. The pharmaceutical composition of claim 9, wherein the at least one systemically active agent is an anti-Alzheimer drug selected from the group consisting of choline esterase inhibitors such as tacrine, donepezil, rivastigmine, galantamine, amantadine, pharmaceutically acceptable salts, isomers and pro-drugs thereof, and mixtures thereof.

12. The pharmaceutical composition of claim 9, wherein the at least one systemically active agent is an analgesics drug selected from the group consisting of alfentanil, buprenorphine, butorphanol, codeine, dextromoramide, dextropropoxyphene, dezocine, diamorphine, dihydrocodeine, fentanyl, flupirtine, hydrocodone, hydromorphone, ketobemidone, levomethadyl, mepiridine, meptazinol, methadone, morphine, nalbuphine, oxycodone, papaveretum, pentazocine, pethidine, phenoperidine, piritramide, remifentanil, tilidine, tramadol, sufentanil pharmaceutically acceptable salts, isomers and pro-drugs thereof, and mixtures thereof.

13. The pharmaceutical composition of claim 9, wherein the at least one systemically active agent is an anti-addiction drug selected from the group consisting of nicotine, buprenorphine, naloxone, pharmaceutically acceptable salts, isomers and pro-drugs thereof, and mixtures thereof.

14. The pharmaceutical composition of claim 9, wherein the at least one systemically active agent is an anti-psychotic drug selected from the group consisting of phenothiazines such as chlorpromazine, fluphenazine, perphenazine, prochlorperazine, thioridazine, trifluoperazine; butyrophenones such haloperidol, droperidol, pimozide; clozapine, olanzapine, mirtanzapine, tinaeptine, bupropion, risperidone, quetiapine, ziprasidone, amisulpride, melperone, paliperidone, aripiprazole, pharmaceutically acceptable salts, isomers and pro-drugs thereof, and mixtures thereof.

15. The pharmaceutical composition of claim 9, wherein the at least one systemically active agent is an anti-anxiety drug selected from the group consisting of benzodiazepines such as alprazolam, bromazepam, diazepam, lorazepam, clonazepam, temazepam, oxazepam, flunitrazepam, triazolam, chlordiazepoxide, flurazepam, estazolam, nitrazepam, pharmaceutically acceptable derivatives such as salts and isomers thereof, pharmaceutically acceptable pro-drugs thereof, and mixtures thereof.

16. The pharmaceutical composition of claim 9, wherein the at least one systemically active agent is an anti-depressant drug selected from the group consisting of selective serotonine reuptake inhibitors (SSRIs) as citalopram, escitalopram oxalate, fluoxetine, fluvoxamine, paroxetine, sertraline, dapoxetine; serotonin-norepinephrine reuptake inhibitors (SNRIs) such as venlafaxine and duloxetine; monoamine oxidase inhibitors (MAOI) such as harmaline, iproniazid, isocarboxazid, nialamide, pargyline, phenelzine, selegiline, toloxatone, tranylcypromine,brofaromine, moclobemide; tricyclic anti-depressants such as amitriptyline, amoxapine, butriptyline, clomipramine, desipramine, dibenzepin, dothiepin, doxepin, imipramine, iprindole, lofepramine, melitracen, nortriptyline, opipramol, protriptyline, trimipramine; tetracyclic anti-depressants such as maprotiline, mianserin, nefazodone, trazodone, pharmaceutically acceptable derivatives such as salts and isomers thereof, pharmaceutically acceptable pro-drugs thereof, and mixtures thereof.

17. The pharmaceutical composition of claim 9, wherein the at least one systemically active agent is an anti-insomnia drug selected from the group consisting of zolpidem, zopiclone, pharmaceutically acceptable salts, isomers and pro-drugs thereof, and mixtures thereof.

18. The pharmaceutical composition of claim 9, wherein the at least one systemically active agent is a drug for treating ADHD selected from the group consisting of methylphenidate, pharmaceutically acceptable salts, isomers and pro-drugs thereof, and mixtures thereof.

19. The pharmaceutical formulation of claim 1, further comprising an agent selected from the group consisting of gelling agents; permeation enhancers, preservatives, anti-oxidants, buffers, humectants, sequestering agents, moisturizers, surfactants, emollients, film-forming agents, solubilizers, flavors, fragrances, stabilizers, solubilizers, and any combination thereof.

20. A method of delaying or inhibiting crystallization of a systemically active agent in a transdermal or transmucosal pharmaceutical formulation according to claim 1 when applied to the skin or mucosal surface of a mammal.
